(19) United States
(12) Patent Application Publication
(10) Pub. No.: US 2018/0200040 A1
(43) Pub. Date: Jul. 19, 2018

(54) EMBOLIC PROTECTION DEVICE

(71) Applicant: BOSTON SCIENTIFIC SCIMED INC., Maple Grove, MN (US)

(72) Inventors: Joel M. WasDyke, Eden Prairie, MN (US); Patrick A. Haverkost, Corcoran, MN (US)

(73) Assignee: BOSTON SCIENTIFIC SCIMED INC., Maple Grove, MN (US)

(21) Appl. No.: 15/874,002

(22) Filed: Jan. 18, 2018

Related U.S. Application Data
(60) Provisional application No. 62/447,646, filed on Jan. 18, 2017.

(51) Int. Cl. A61F 2/01; A61B 17/221

(52) U.S. Cl. CPC A61F 2/013; A61B 2017/00862; A61B 17/221

(57) ABSTRACT

An embolic protection system may include a guidewire having a length, at least a portion of the guidewire having a non-circular cross-sectional shape, and an embolic protection device including a mounting sleeve configured to attach the embolic protection device to the portion of the guidewire having the non-circular cross-sectional shape. The mounting sleeve may include a non-circular interior cross-sectional shape and a non-circular exterior cross-sectional shape.

### CROSS-REFERENCE TO RELATED APPLICATIONS

This application claims the benefit of priority under 35 U.S.C. § 119 to U.S. Provisional Application Ser. No. 62/447,646, filed Jan. 18, 2017, the entirety of which is incorporated herein by reference.

### TECHNICAL FIELD

The present disclosure pertains to devices for embolic and/or distal protection used to capture percutaneous debris and/or embolic material.

### BACKGROUND

A wide variety of intracorporeal medical devices have been developed for medical use, for example, intravascular use. Some of these devices include guidewires, embolic protection devices, distal protection filters, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

In a first aspect, an embolic protection system may comprise a guidewire having a length, at least a portion of the guidewire having a non-circular cross-sectional shape, and an embolic protection device including a mounting sleeve configured to attach the embolic protection device to the portion of the guidewire having the non-circular cross-sectional shape. The mounting sleeve may include a non-circular interior cross-sectional shape and a non-circular exterior cross-sectional shape.

In addition or alternatively, and in a second aspect, the non-circular interior cross-sectional shape and the non-circular exterior cross-sectional shape are substantially identical.

In addition or alternatively, and in a third aspect, the embolic protection device is slidable with respect to the guidewire.

In addition or alternatively, and in a fourth aspect, the embolic protection device is non-rotatable with respect to the guidewire.

In addition or alternatively, and in a fifth aspect, the embolic protection system may further comprise a delivery catheter having a lumen extending therethrough.

In addition or alternatively, and in a sixth aspect, the embolic protection device is disposed within a distal portion of the lumen in a delivery configuration.

In addition or alternatively, and in a seventh aspect, the embolic protection device is expandable to a deployed configuration when the embolic protection device is disposed outside of the lumen.

In addition or alternatively, and in an eighth aspect, the embolic protection device includes a second mounting sleeve configured to attach to the portion of the guidewire having the non-circular cross-sectional shape.

In addition or alternatively, and in a ninth aspect, the second mounting sleeve includes the non-circular interior cross-sectional shape of the mounting sleeve and the non-circular exterior cross-sectional shape of the mounting sleeve.

In addition or alternatively, and in a tenth aspect, the mounting sleeve is fixed to the embolic protection device proximate a proximal mouth of the embolic protection device and the second mounting sleeve is fixed to the embolic protection device proximate a distal end of the embolic protection device.

In addition or alternatively, and in an eleventh aspect, the mounting sleeve and the second mounting sleeve define a distance from a proximal end of the mounting sleeve to a distal end of the second mounting sleeve. The portion of the guidewire having the non-circular cross-sectional shape may be longer than the distance from the proximal end of the mounting sleeve to the distal end of the second mounting sleeve and less than the length of the guidewire.

In addition or alternatively, and in a twelfth aspect, the embolic protection system may further comprise a proximal stop disposed on the guidewire proximal of the mounting sleeve and a distal stop disposed on the guidewire distal of the mounting sleeve.

In addition or alternatively, and in a thirteenth aspect, the proximal stop is fixedly attached to the guidewire proximate a proximal end of the portion of the guidewire having the non-circular cross-sectional shape.

In addition or alternatively, and in a fourteenth aspect, the distal stop is fixedly attached to the guidewire proximate a distal end of the portion of the guidewire having the non-circular cross-sectional shape.

In addition or alternatively, and in a fifteenth aspect, the embolic protection device includes a filter element and at least one support strut.

In addition or alternatively, and in a sixteenth aspect, an embolic protection system may comprise a guidewire having a length, at least a portion of the guidewire having a polygonal cross-sectional shape, and an embolic protection device including a mounting sleeve configured to attach the embolic protection device to the portion of the guidewire having the polygonal cross-sectional shape. The mounting sleeve may include a polygonal interior cross-sectional shape and a polygonal exterior cross-sectional shape.

In addition or alternatively, and in a seventeenth aspect, the polygonal interior cross-sectional shape and the polygonal exterior cross-sectional shape are substantially identical.

In addition or alternatively, and in an eighteenth aspect, the embolic protection device includes a second mounting sleeve configured to attach to the portion of the guidewire having the polygonal cross-sectional shape.

In addition or alternatively, and in a nineteenth aspect, the second mounting sleeve includes the polygonal interior cross-sectional shape of the mounting sleeve and the polygonal exterior cross-sectional shape of the mounting sleeve.

In addition or alternatively, and in a twentieth aspect, an embolic protection system may comprise a guidewire having a length, at least a portion of the guidewire having a non-circular cross-sectional shape, and an embolic protection device including a filter element, a mounting sleeve, and a second mounting sleeve. The mounting sleeve and the second mounting sleeve may be configured to slidably attach the embolic protection device to the portion of the guidewire having the non-circular cross-sectional shape. The mounting sleeve and the second mounting sleeve may each include a non-circular interior cross-sectional shape and a non-circular exterior cross-sectional shape. Axial movement of the embolic protection device relative to the guidewire may be limited to less than ten percent of the length of the guidewire.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 illustrates a partial cross-sectional view of an example embolic protection system in a delivery configuration;

FIG. 2 illustrates a partial cross-sectional view of an example embolic protection system in a deployed configuration;

FIGS. 3-5 are cross-sectional views taken at line A-A of FIG. 2 showing example configurations of an embolic protection device;

FIG. 6 illustrates a partial cross-sectional view of an example embolic protection system in a deployed configuration; and

FIGS. 7-9 are cross-sectional view taken at line B-B of FIG. 6 showing example configurations of an embolic protection device.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the claimed invention. Those skilled in the art will recognize that the various elements described and/or shown may be arranged in various combinations and configurations without departing from the scope of the disclosure. The detailed description and drawings illustrate example embodiments of the claimed invention.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g. 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosed invention are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a measurement of a stated or identified dimension or feature. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, a maximum “extent” may be considered a greatest possible dimension measured according to the intended usage. In some instances, a “minimum” extent may refer to a smallest possible measurement of a stated or identified dimension according to the intended usage. Such instances will be readily apparent to the skilled person from the context of the individual usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

This disclosure pertains to devices or systems for deploying an embolic protection device, or other device(s) as described herein, in a bodily passageway and/or lumen. Deployment may be achieved for medical applications in the cardiovascular system (e.g., in the heart, veins, and/or arteries), in the neurological system (e.g., in the brain), and/or in the gastrointestinal tract, the biliary tract, the urinary tract, and/or the respiratory tract, as appropriate. Reference to bodily passageways and/or lumens may be to passageways and/or lumens in any of the aforementioned tracts and systems or elsewhere in the body.

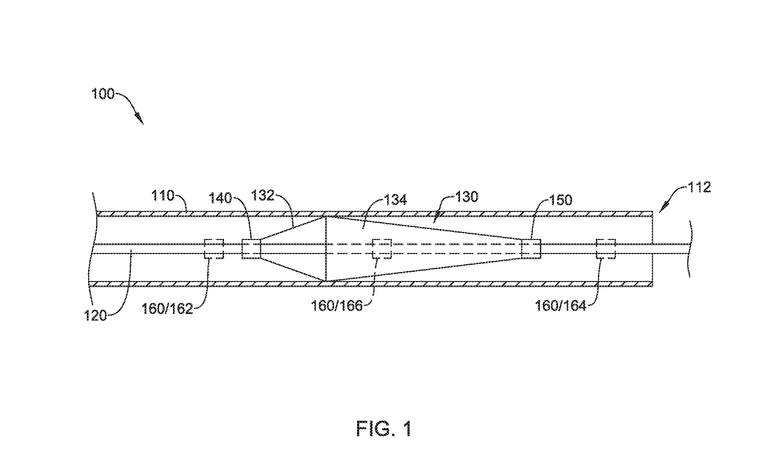

FIG. 1 illustrates an embolic protection system 100 comprising a delivery catheter 110 having a lumen 112 extending therethrough, a guidewire 120 slidably disposed within the lumen of the delivery catheter 110, and an embolic protection device 130 disposed within a distal portion of the lumen of the delivery catheter 110 in a delivery configuration. The guidewire 120 has a length, and in some embodiments, at least a portion of the length of the guidewire 120 may have a non-circular cross-sectional shape. In some embodiments, at least a portion of the length of the guidewire 120 may have a polygonal cross-sectional shape. In some embodiments, the portion of the length of the guidewire 120 having the non-circular cross-sectional shape and/or the polygonal cross-sectional shape may be formed using a variety of suitable means including, but not limited to, grinding, stamping, electro-polishing, roll forming, electro-discharge machining (EDM), etc. Some suitable but non-limiting materials for the delivery catheter 110 and/or the guidewire 120, for example metallic and/or polymeric materials, are described below.

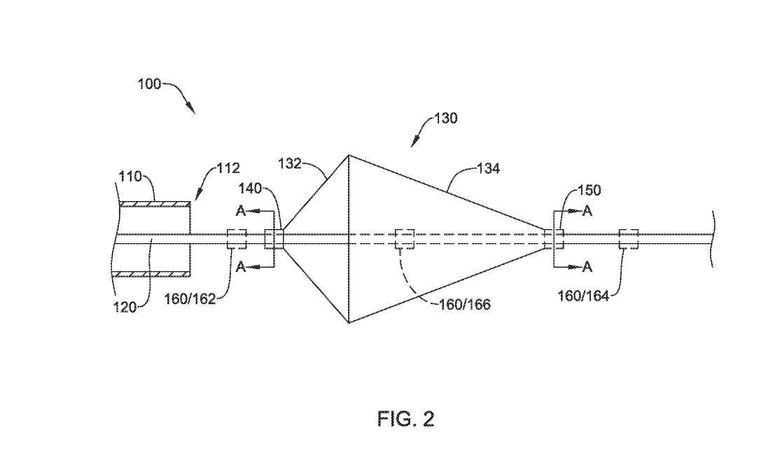

In some embodiments, the embolic protection device 130 may be slidable with respect to the guidewire 120. In some embodiments, the embolic protection device 130 may be non-rotatable with respect to the guidewire 120. In some embodiments, the embolic protection device 130 may be expandable to a deployed configuration when the embolic protection device 130 is disposed outside of the lumen of the delivery catheter 110 and/or when the embolic protection device 130 is unconstrained by the delivery catheter 110, as seen in FIG. 2 for example. In some embodiments, the embolic protection device 130 may include a filter element 134 and at least one support strut 132. In some embodiments, the at least one support strut 132 may extend proximally from the filter element 134. In some embodiments, the filter element 134 may be disposed on, over, and/or encapsulate the at least one support strut 132 and/or a portion of the at least one support strut 132. In some embodiments, the at least one support strut 132 may be connected to and/or integrally formed with a support hoop forming a proximal mouth of the embolic protection device 130 and/or the filter element 134. In at least some embodiments, the support hoop may be omitted. In some embodiments, the embolic protection device 130 and/or the filter element 134 may have a closed distal end and/or be configured to capture and/or retain embolic material and/or debris therein.

In at least some embodiments, the filter element 134 may include and/or be formed with a plurality of pores, apertures, and/or openings configured to filter embolic material and/or debris from a fluid (e.g., blood) passing through the filter element 134. In some embodiments, the filter element 134 may be formed from a mesh, a woven material, a textile, etc. In some embodiments, the filter element 134 may be formed from a polymeric and/or metallic film and/or membrane having the plurality of pores, apertures, and/or openings formed therein. Other suitable configurations and/or constructions are also contemplated. In some embodiments, the filter element 134 may be configured to filter embolic material and/or debris having a size greater than the plurality of pores, apertures, and/or openings formed in the filter element 134 and capture and/or retain the embolic material and/or debris therein. Some suitable but non-limiting materials for the embolic protection device 130, the at least one support strut 132, and/or the filter element 134, for example metallic and/or polymeric materials, are described below.

In some embodiments, the embolic protection device 130 may include a mounting sleeve 140 configured to attach the embolic protection device 130 to the portion of the guidewire 120 having the non-circular cross-sectional shape and/or the polygonal cross-sectional shape. In some embodiments, the mounting sleeve 140 is configured to slidably attach the embolic protection device 130 to the portion of the guidewire 120 having the non-circular cross-sectional shape and/or the polygonal cross-sectional shape. For example, the mounting sleeve 140 may be slidable with respect to at least a portion of the guidewire 120, for example, the portion of the guidewire 120 having the non-circular cross-sectional shape and/or the polygonal cross-sectional shape. The mounting sleeve 140 may be fixed to the embolic protection device 130 proximate a proximal mouth of the embolic protection device 130.

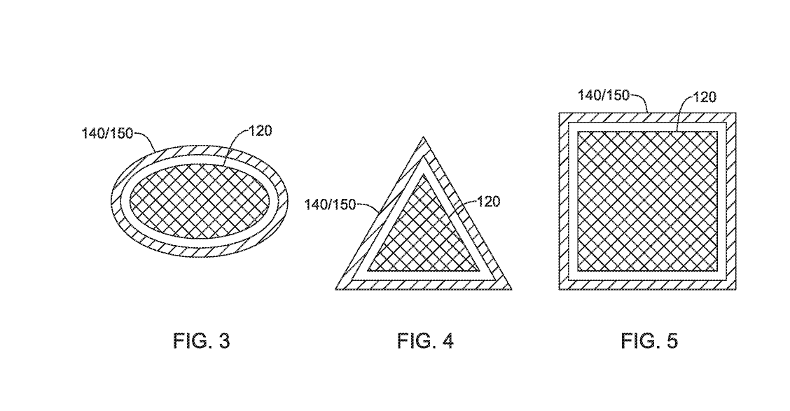

In at least some embodiments, the mounting sleeve 140 may include a non-circular interior cross-sectional shape and a non-circular exterior cross-sectional shape. FIGS. 3-5 illustrate example non-circular interior cross-sectional shapes of mounting sleeve 140 taken along line A-A of FIG. 2. For example, the mounting sleeve 140 may include an oval cross-sectional shape as seen in FIG. 3, a triangular cross-sectional shape as seen in FIG. 4, or a square cross sectional shape as seen in FIG. 5. FIGS. 3-5 are provided as non-limiting examples of non-circular cross-sectional shapes of the mounting sleeve 140. In some embodiments, the non-circular interior cross-sectional shape of the mounting sleeve 140 and the non-circular exterior cross-sectional shape of the mounting sleeve 140 may be substantially identical. Alternatively, in some embodiments, the non-circular interior cross-sectional shape of the mounting sleeve 140 and the non-circular exterior cross-sectional shape of the mounting sleeve 140 may be different from each other. In at least some embodiments, the mounting sleeve 140 may include a polygonal interior cross-sectional shape and a polygonal exterior cross-sectional shape. In some embodiments, the polygonal interior cross-sectional shape of the mounting sleeve 140 and the polygonal exterior cross-sectional shape of the mounting sleeve 140 may be substantially identical. Alternatively, in some embodiments, the polygonal interior cross-sectional shape of the mounting sleeve 140 and the polygonal exterior cross-sectional shape of the mounting sleeve 140 may be different from each other. In some embodiments, the mounting sleeve 140 may include the non-circular interior cross-sectional shape and/or the polygonal interior cross-sectional shape, and a circular exterior cross-sectional shape. Other configurations are also contemplated.

In some embodiments, the mounting sleeve 140 is configured to non-rotatably or rotatably attach the embolic protection device 130 to the portion of the guidewire 120 having the non-circular cross-sectional shape and/or the polygonal cross-sectional shape. For example, the mounting sleeve 140 may be non-rotatable with respect to at least a portion of the guidewire 120, for example, the portion of the guidewire 120 having the non-circular cross-sectional shape and/or the polygonal cross-sectional shape. Some suitable but non-limiting materials for the mounting sleeve 140, for example metallic and/or polymeric materials, are described below.

In some embodiments, the embolic protection device 130 may include a second mounting sleeve 150 configured to attach the embolic protection device 130 to the portion of the guidewire 120 having the non-circular cross-sectional shape and/or the polygonal cross-sectional shape. In some embodiments, the second mounting sleeve 150 is configured to slidably attach the embolic protection device 130 to the portion of the guidewire 120 having the non-circular cross-sectional shape and/or the polygonal cross-sectional shape. For example, the second mounting sleeve 150 may be slidable with respect to at least a portion of the guidewire 120, for example, the portion of the guidewire 120 having the non-circular cross-sectional shape and/or the polygonal cross-sectional shape. The second mounting sleeve 150 may be fixed to the embolic protection device 130 proximate a distal end of the embolic protection device 130.

In at least some embodiments, the second mounting sleeve 150 may include a non-circular interior cross-sectional shape and a non-circular exterior cross-sectional shape. FIGS. 3-5 illustrate example non-circular interior cross-sectional shapes of mounting sleeve 150 taken along line A-A of FIG. 2. For example, the second mounting sleeve 150 may include an oval cross-sectional shape as seen in FIG. 3, a triangular cross-sectional shape as seen in FIG. 4, or a square cross sectional shape as seen in FIG. 5. FIGS. 3-5 are provided as non-limiting examples of non-circular cross-sectional shapes of the second mounting sleeve 150. In some embodiments, the second mounting sleeve 150 may include the non-circular interior cross-sectional shape of the mounting sleeve 140 and the non-circular exterior cross-sectional shape of the mounting sleeve 140. In some embodiments, the non-circular interior cross-sectional shape of the second mounting sleeve 150 and the non-circular exterior cross-sectional shape of the second mounting sleeve 150 may be substantially identical. Alternatively, in some embodiments, the non-circular interior cross-sectional shape of the second mounting sleeve 150 and the non-circular exterior cross-sectional shape of the second mounting sleeve 150 may be different from each other. In at least some embodiments, the second mounting sleeve 150 may include a polygonal interior cross-sectional shape and a polygonal exterior cross-sectional shape. In some embodiments, the second mounting sleeve 150 may include the polygonal interior cross-sectional shape of the mounting sleeve 140 and the polygonal exterior cross-sectional shape of the mounting sleeve 140. In some embodiments, the polygonal interior cross-sectional shape of the second mounting sleeve 150 and the polygonal exterior cross-sectional shape of the second mounting sleeve 150 may be substantially identical. Alternatively, in some embodiments, the polygonal interior cross-sectional shape of the second mounting sleeve 150 and the polygonal exterior cross-sectional shape of the second mounting sleeve 150 may be different from each other. In some embodiments, the second mounting sleeve 150 may include the non-circular interior cross-sectional shape and/or the polygonal interior cross-sectional shape, and a circular exterior cross-sectional shape. Other configurations are also contemplated.

In some embodiments, the second mounting sleeve 150 is configured to non-rotatably or rotatably attach the embolic protection device 130 to the portion of the guidewire 120 having the non-circular cross-sectional shape and/or the polygonal cross-sectional shape. For example, the second mounting sleeve 150 may be non-rotatable with respect to at least a portion of the guidewire 120, for example, the portion of the guidewire 120 having the non-circular cross-sectional shape and/or the polygonal cross-sectional shape.

In embodiments having the mounting sleeve 140 and the second mounting sleeve 150, the mounting sleeve 140 and the second mounting sleeve 150 may have, but are not required to have, the same non-circular and/or polygonal interior cross-sectional shape. Similarly, in embodiments having the mounting sleeve 140 and the second mounting sleeve 150, the mounting sleeve 140 and the second mounting sleeve 150 may have, but are not required to have, the same non-circular and/or polygonal exterior cross-sectional shape. Various combinations of shapes may be used among the mounting sleeve 140 and the second mounting sleeve 150 if they are compatible with the portion of the guidewire 120 having the non-circular cross-sectional shape and/or the polygonal cross-sectional shape. Some suitable but non-limiting materials for the second mounting sleeve 150, for example metallic and/or polymeric materials, are described below.

In some embodiments, the mounting sleeve 140 and/or the second mounting sleeve 150 may include a split and/or an open channel oriented longitudinally along a length of the mounting sleeve 140 and/or the second mounting sleeve 150. In these embodiments, the mounting sleeve 140 and/or the second mounting sleeve 150 may permit the embolic protection device 130 to rotate about the guidewire 120 in a ratcheting manner if enough relative rotational force is applied to the guidewire 120 and/or the embolic protection device 130. When enough relative rotational force is applied to the guidewire 120 and/or the embolic protection device 130, the mounting sleeve 140 and/or the second mounting sleeve 150 may deform, open, and/or spread apart at the split and/or the open channel, thereby permitting the guidewire 120 to rotate within the mounting sleeve 140 and/or the second mounting sleeve 150.

Turning back to FIGS. 1 and 2, the mounting sleeve 140 and the second mounting sleeve 150 may define a distance from a proximal end of the mounting sleeve 140 to a distal end of the second mounting sleeve 150. In at least some embodiments, the portion of the guidewire 120 having the non-circular cross-sectional shape and/or the polygonal cross-sectional shape may be longer than the distance from the proximal end of the mounting sleeve 140 to the distal end of the second mounting sleeve 150. In some embodiments, the portion of the guidewire 120 having the non-circular cross-sectional shape and/or the polygonal cross-sectional shape may permit axial movement of the embolic protection device 130, the mounting sleeve 140, and/or the second mounting sleeve 150 along and/or relative to the guidewire 120. In some embodiments, axial movement of the embolic protection device 130, the mounting sleeve 140, and/or the second mounting sleeve 150 along and/or relative to the guidewire 120 may be limited to less than twenty percent of the length of the guidewire 120. In some embodiments, axial movement of the embolic protection device 130, the mounting sleeve 140, and/or the second mounting sleeve 150 along and/or relative to the guidewire 120 may be limited to less than ten percent of the length of the guidewire 120. In some embodiments, axial movement of the embolic protection device 130, the mounting sleeve 140, and/or the second mounting sleeve 150 along and/or relative to the guidewire 120 may be limited to less than five percent of the length of the guidewire 120. In some embodiments, axial movement of the embolic protection device 130, the mounting sleeve 140, and/or the second mounting sleeve 150 along and/or relative to the guidewire 120 may be limited to less than two percent of the length of the guidewire 120.

In some embodiments, the embolic protection system 100 may optionally include at least one stop 160 disposed on the guidewire 120, as seen in FIGS. 1 and 2. In some embodiments, the at least one stop 160 may be fixedly attached to the guidewire 120. The at least one stop 160 may be configured to limit axial movement of the embolic protection device 130, the mounting sleeve 140, and/or the second mounting sleeve 150 along and/or relative to the guidewire 120 in a proximal direction, a distal direction, or both proximal and distal directions. In some embodiments, the at least one stop 160 may be positioned proximal of the mounting sleeve 140, distal of the mounting sleeve 140, proximal of the second mounting sleeve 150, distal of the second mounting sleeve 150, and/or between the mounting sleeve 140 and the second mounting sleeve 150.

In some embodiments, the at least one stop 160 may include a proximal stop 162 disposed on the guidewire 120 proximal of the mounting sleeve 140 and a distal stop 164 disposed distal of the mounting sleeve 140. In some embodiments, the at least one stop 160 may include a proximal stop 162 disposed on the guidewire 120 proximal of the mounting sleeve 140 and a distal stop 164 disposed distal of the second mounting sleeve 150. In some embodiments, the at least one stop 160 may further include an intermediate stop 166 disposed between the mounting sleeve 140 and the second mounting sleeve 150. Some suitable but non-limiting materials for the at least one stop 160, the proximal stop 162, the distal stop 164, and/or the intermediate stop 166, for example metallic and/or polymeric materials, are described below.

In some embodiments, the at least one stop 160 and/or the proximal stop 162 may be fixedly attached to the guidewire 120 proximate a proximal end of the portion of the guidewire 120 having the non-circular cross-sectional shape and/or the polygonal cross-sectional shape. In some embodiments, the at least one stop 160 and/or the distal stop 164 may be fixedly attached to the guidewire 120 proximate a distal end of the portion of the guidewire 120 having the non-circular cross-sectional shape and/or the polygonal cross-sectional shape. In some embodiments, the at least one stop 160 and/or the intermediate stop 166 may be fixedly attached to the guidewire 120 between the proximal end and the distal end of the portion of the guidewire 120 having the non-circular cross-sectional shape and/or the polygonal cross-sectional shape.

In some embodiments, the mounting sleeve 140 and/or a proximal end of the embolic protection device 130 may be axially constrained along and/or relative to the guidewire 120, and the second mounting sleeve 150 and/or a distal end of the embolic protection device 130 may be axially constrained along and/or relative to the guidewire 120. In some embodiments, the mounting sleeve 140 and/or a proximal end of the embolic protection device 130 may be axially constrained along and/or relative to the guidewire 120, and the second mounting sleeve 150 and/or a distal end of the embolic protection device 130 may be axially unconstrained along and/or relative to the guidewire 120. Leaving the second mounting sleeve 150 and/or the distal end of the embolic protection device 130 axially unconstrained may permit the second mounting sleeve 150 and/or the distal end of the embolic protection device 130 to stretch distally to provide a lower overall profile during sheathing, delivery, and/or retrieval.

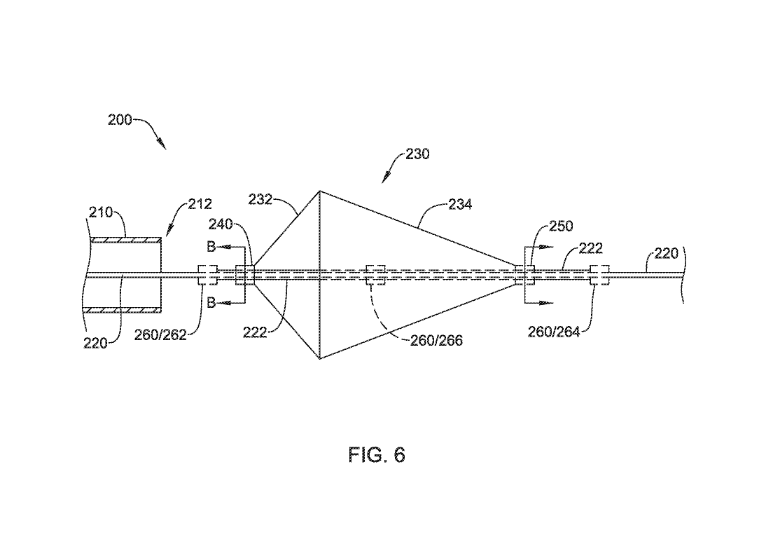

FIG. 6 illustrates an alternative embolic protection system 200, similar in many respects to the embolic protection system 100 above, comprising a delivery catheter 210 having a lumen 212 extending therethrough, a guidewire 220 slidably disposed within the lumen of the delivery catheter 210, and an embolic protection device 230. Although not expressly shown, the embolic protection device 230 may be disposed within a distal portion of the lumen of the delivery catheter 210 in a delivery configuration, similar to the embodiment of FIG. 1 above. In some embodiments, the guidewire 220 may have a round or circular cross-sectional shape and/or profile. The guidewire 220 has a length, and in some embodiments, and at least a portion of the length of the guidewire 220 may include a polymer sleeve 222 disposed on and/or about the guidewire 220, the polymer sleeve 222 having a length and a non-circular cross-sectional shape extending along at least a portion of the length of the polymer sleeve 222. In some embodiments, the polymer sleeve 222 may be fixedly attached to the guidewire 220. In some embodiments, the polymer sleeve 222 may be secured to the guidewire 220 by one or more of a variety of suitable means, including but not limited to, interference fit, press fit, friction fit, adhesive bonding, etc. In some embodiments, at least a portion of the length of the polymer sleeve 222 may have a polygonal cross-sectional shape extending along at least a portion of the length of the polymer sleeve 222. In some embodiments, the portion of the length of the polymer sleeve 222 having the non-circular cross-sectional shape and/or the polygonal cross-sectional shape may be formed using a variety of suitable means including, but not limited to, molding, extrusion, machining, etc. Some suitable but non-limiting materials for the delivery catheter 210, the guidewire 220, and/or the polymer sleeve 222, for example metallic and/or polymeric materials, are described below.

In some embodiments, the embolic protection device 230 may be slidable with respect to the guidewire 220 and/or the polymer sleeve 222. In some embodiments, the embolic protection device 230 may be non-rotatable with respect to the guidewire 220 and/or the polymer sleeve 222. In some embodiments, the embolic protection device 230 may be expandable to a deployed configuration when the embolic protection device 230 is disposed outside of the lumen of the delivery catheter 210 and/or when the embolic protection device 130 is unconstrained by the delivery catheter 210, as seen in FIG. 6 for example. In some embodiments, the embolic protection device 230 may include a filter element 234 and at least one support strut 232. In some embodiments, the at least one support strut 232 may extend proximally from the filter element 234. In some embodiments, the filter element 234 may be disposed on, over, and/or encapsulate the at least one support strut 232 and/or a portion of the at least one support strut 232. In some embodiments, the at least one support strut 232 may be connected to and/or integrally formed with a support hoop forming a proximal mouth of the embolic protection device 230 and/or the filter element 234. In at least some embodiments the support hoop may be omitted. In some embodiments, the embolic protection device 230 and/or the filter element 234 may have a closed distal end and/or be configured to capture and/or retain embolic material and/or debris therein.

In at least some embodiments, the filter element 234 may include and/or be formed with a plurality of pores, apertures, and/or openings configured to filter embolic material and/or debris from a fluid (e.g., blood) passing through the filter element 234. In some embodiments, the filter element 234 may be formed from a mesh, a woven material, a textile, etc. In some embodiments, the filter element 234 may be formed from a polymeric and/or metallic film and/or membrane having the plurality of pores, apertures, and/or openings formed therein. Other suitable configurations and/or constructions are also contemplated. In some embodiments, the filter element 234 may be configured to filter embolic material and/or debris having a size greater than the plurality of pores, apertures, and/or openings formed in the filter element 234 and capture and/or retain the embolic material and/or debris therein. Some suitable but non-limiting materials for the embolic protection device 230, the at least one support strut 232, and/or the filter element 234, for example metallic and/or polymeric materials, are described below.

In some embodiments, the embolic protection device 230 may include a mounting sleeve 240 configured to attach the embolic protection device 230 to the polymer sleeve 222. In some embodiments, the mounting sleeve 240 is configured to slidably attach the embolic protection device 230 to the polymer sleeve 222. For example, the mounting sleeve 240 may be slidable with respect to at least a portion of the guidewire 220, for example, the polymer sleeve 222 having the non-circular cross-sectional shape and/or the polygonal cross-sectional shape extending along at least a portion of the length of the polymer sleeve 222. The mounting sleeve 240 may be fixed to the embolic protection device 230 proximate a proximal mouth of the embolic protection device 230.

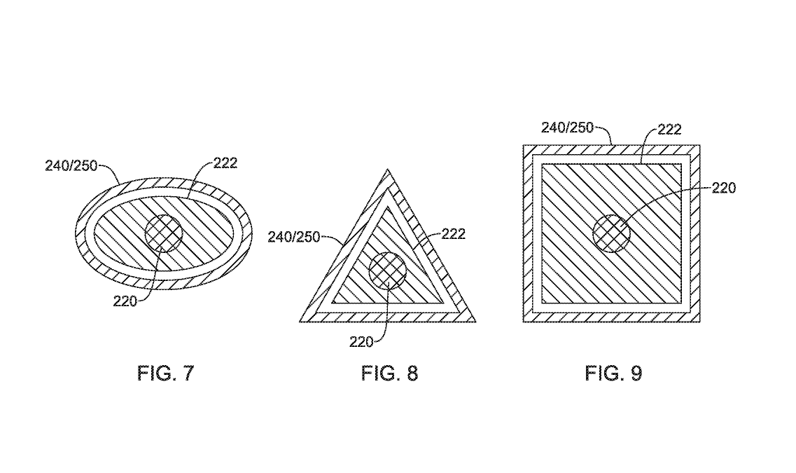

In at least some embodiments, the mounting sleeve 240 may include a non-circular interior cross-sectional shape and a non-circular exterior cross-sectional shape. FIGS. 7-9 illustrate example non-circular interior cross-sectional shapes of mounting sleeve 240 taken along line B-B of FIG. 6. For example, the mounting sleeve 240 may include an oval cross-sectional shape as seen in FIG. 7, a triangular cross-sectional shape as seen in FIG. 8, or a square cross sectional shape as seen in FIG. 9. FIGS. 7-9 are provided as non-limiting examples of non-circular cross-sectional shapes of the mounting sleeve 240. In some embodiments, the non-circular interior cross-sectional shape of the mounting sleeve 240 and the non-circular exterior cross-sectional shape of the mounting sleeve 240 may be substantially identical. Alternatively, in some embodiments, the non-circular interior cross-sectional shape of the mounting sleeve 240 and the non-circular exterior cross-sectional shape of the mounting sleeve 240 may be different from each other. In at least some embodiments, the mounting sleeve 240 may include a polygonal interior cross-sectional shape and a polygonal exterior cross-sectional shape. In some embodiments, the polygonal interior cross-sectional shape of the mounting sleeve 240 and the polygonal exterior cross-sectional shape of the mounting sleeve 240 may be substantially identical. Alternatively, in some embodiments, the polygonal interior cross-sectional shape of the mounting sleeve 240 and the polygonal exterior cross-sectional shape of the mounting sleeve 240 may be different from each other. In some embodiments, the mounting sleeve 240 may include the non-circular interior cross-sectional shape and/or the polygonal interior cross-sectional shape, and a circular exterior cross-sectional shape. Other configurations are also contemplated.

In some embodiments, the mounting sleeve 240 is configured to non-rotatably or rotabably attach the embolic protection device 230 to the guidewire 220 and/or the polymer sleeve 222 having the non-circular cross-sectional shape and/or the polygonal cross-sectional shape extending along at least a portion of the length of the polymer sleeve 222. For example, the mounting sleeve 240 may be non-rotatable with respect to at least a portion of the guidewire 220, for example, the polymer sleeve 222 having the non-circular cross-sectional shape and/or the polygonal cross-sectional shape extending along at least a portion of the length of the polymer sleeve 222. In some embodiments, the non-circular interior cross-sectional shape and/or the polygonal interior cross-sectional shape of the mounting sleeve 240 may correspond to the non-circular cross-sectional shape extending along at least a portion of the polymer sleeve 222. Some suitable but non-limiting materials for the mounting sleeve 240, for example metallic and/or polymeric materials, are described below.

In some embodiments, the embolic protection device 230 may include a second mounting sleeve 250 configured to attach the embolic protection device 230 to the polymer sleeve 222. In some embodiments, the second mounting sleeve 250 is configured to slidably attach the embolic protection device 230 to the polymer sleeve 222. For example, the second mounting sleeve 250 may be slidable with respect to at least a portion of the guidewire 220, for example, the polymer sleeve 222 having the non-circular cross-sectional shape and/or the polygonal cross-sectional shape extending along at least a portion of the length of the polymer sleeve 222. The second mounting sleeve 250 may be fixed to the embolic protection device 230 proximate a distal end of the embolic protection device 230.

In at least some embodiments, the second mounting sleeve 250 may include a non-circular interior cross-sectional shape and a non-circular exterior cross-sectional shape. FIGS. 7-9 illustrate example non-circular interior cross-sectional shapes of mounting sleeve 250 taken along line B-B of FIG. 6. For example, the second mounting sleeve 250 may include an oval cross-sectional shape as seen in FIG. 7, a triangular cross-sectional shape as seen in FIG. 8, or a square cross sectional shape as seen in FIG. 9. FIGS. 7-9 are provided as non-limiting examples of non-circular cross-sectional shapes of the second mounting sleeve 250. In some embodiments, the second mounting sleeve 250 may include the non-circular interior cross-sectional shape of the mounting sleeve 240 and the non-circular exterior cross-sectional shape of the mounting sleeve 240. In some embodiments, the non-circular interior cross-sectional shape of the second mounting sleeve 250 and the non-circular exterior cross-sectional shape of the second mounting sleeve 250 may be substantially identical. Alternatively, in some embodiments, the non-circular interior cross-sectional shape of the second mounting sleeve 250 and the non-circular exterior cross-sectional shape of the second mounting sleeve 250 may be different from each other. In at least some embodiments, the second mounting sleeve 250 may include a polygonal interior cross-sectional shape and a polygonal exterior cross-sectional shape. In some embodiments, the second mounting sleeve 250 may include the polygonal interior cross-sectional shape of the mounting sleeve 240 and the polygonal exterior cross-sectional shape of the mounting sleeve 240. In some embodiments, the polygonal interior cross-sectional shape of the second mounting sleeve 250 and the polygonal exterior cross-sectional shape of the second mounting sleeve 250 may be substantially identical. Alternatively, in some embodiments, the polygonal interior cross-sectional shape of the second mounting sleeve 250 and the polygonal exterior cross-sectional shape of the second mounting sleeve 250 may be different from each other. In some embodiments, the second mounting sleeve 250 may include the non-circular interior cross-sectional shape and/or the polygonal interior cross-sectional shape, and a circular exterior cross-sectional shape. Other configurations are also contemplated.

In some embodiments, the second mounting sleeve 250 is configured to non-rotatably or rotatably attach the embolic protection device 230 to the guidewire 220 and/or the polymer sleeve 222 having the non-circular cross-sectional shape and/or the polygonal cross-sectional shape extending along at least a portion of the length of the polymer sleeve 222. For example, the second mounting sleeve 250 may be non-rotatable with respect to at least a portion of the guidewire 220, for example, the polymer sleeve 222 having the non-circular cross-sectional shape and/or the polygonal cross-sectional shape extending along at least a portion of the length of the polymer sleeve 222. In some embodiments, the non-circular interior cross-sectional shape and/or the polygonal interior cross-sectional shape of the second mounting sleeve 250 may correspond to the non-circular cross-sectional shape and/or the polygonal cross-sectional shape extending along at least a portion of the polymer sleeve 222.

In embodiments having the mounting sleeve 240 and the second mounting sleeve 250, the mounting sleeve 240 and the second mounting sleeve 250 may have, but are not required to have, the same non-circular and/or polygonal interior cross-sectional shape. Similarly, in embodiments having the mounting sleeve 240 and the second mounting sleeve 250, the mounting sleeve 240 and the second mounting sleeve 250 may have, but are not required to have, the same non-circular and/or polygonal exterior cross-sectional shape. Various combinations of shapes may be used among the mounting sleeve 240 and the second mounting sleeve 250 if they are compatible with the portion of the guidewire 220 having the non-circular cross-sectional shape and/or the polygonal cross-sectional shape extending along at least a portion of the polymer sleeve 222. Some suitable but non-limiting materials for the second mounting sleeve 250, for example metallic and/or polymeric materials, are described below.

In some embodiments, the polymer sleeve 222 may be fixedly attached to and/or non-rotatable with respect to the guidewire 220 during normal usage and/or under normal working loads within the patient's anatomy and/or body passageway(s) or lumen(s). As such, during normal usage and/or under normal working loads, the mounting sleeve 240, the second mounting sleeve 250, and/or the embolic protection device 230 may be non-rotatable with respect to the guidewire 220.

In some embodiments, the mounting sleeve 240 and/or the second mounting sleeve 250 may include a split and/or an open channel oriented longitudinally along a length of the mounting sleeve 240 and/or the second mounting sleeve 250. In these embodiments, the mounting sleeve 240 and/or the second mounting sleeve 250 may permit the embolic protection device 230 to rotate about the guidewire 220 and/or the polymer sleeve 222 in a ratcheting manner if enough relative rotational force is applied to the guidewire 220 and/or the embolic protection device 230. When enough relative rotational force is applied to the guidewire 220 and/or the embolic protection device 230, the mounting sleeve 240 and/or the second mounting sleeve 250 may deform, open, and/or spread apart at the split and/or the open channel, thereby permitting the guidewire 220 and/or the polymer sleeve 222 to rotate within the mounting sleeve 240 and/or the second mounting sleeve 250.

In some embodiments, the polymer sleeve 222 may also or alternatively be configured to release and/or break loose from the guidewire 220 when sufficient relative rotational force is applied thereto, thereby permitting the polymer sleeve 222 and/or the embolic protection device 230 to rotate relative to the guidewire 220. For example, if a rotational force exceeding a predetermined limit is applied to the guidewire 220 and the embolic protection device 230 does not rotate along with the guidewire 220 or is prevented from rotating along with the guidewire 220, the polymer sleeve 222 may release from the guidewire 220 when the predetermined limit of rotational force is achieved and/or surpassed, thereby permitting the guidewire 220 to rotate within and/or relative to the polymer sleeve 222 and/or the embolic protection device 230. Such a configuration may be beneficial for preventing the embolic protection device 230 from damaging the wall of the bodily passageway and/or lumen that the embolic protection device 230 is deployed in when the guidewire 220 is subjected to an inadvertently increased or extreme rotational force.

Turning back to FIG. 6, the mounting sleeve 240 and the second mounting sleeve 250 may define a distance from a proximal end of the mounting sleeve 240 to a distal end of the second mounting sleeve 250. In some embodiments, the polymer sleeve 222 may be longer than the distance from the proximal end of the mounting sleeve 240 to the distal end of the second mounting sleeve 250. In at least some embodiments, the non-circular cross-sectional shape and/or the polygonal cross-sectional shape extending along at least a portion of the length of the polymer sleeve 222 may be longer than the distance from the proximal end of the mounting sleeve 240 to the distal end of the second mounting sleeve 250. In some embodiments, the polymer sleeve 222 may permit axial movement of the embolic protection device 230, the mounting sleeve 240, and/or the second mounting sleeve 250 along and/or relative to the guidewire 220 and/or the polymer sleeve 222. In some embodiments, axial movement of the embolic protection device 230, the mounting sleeve 240, and/or the second mounting sleeve 250 along and/or relative to the guidewire 220 and/or the polymer sleeve 222 may be limited to less than twenty percent of the length of the guidewire 220. In some embodiments, axial movement of the embolic protection device 230, the mounting sleeve 240, and/or the second mounting sleeve 250 along and/or relative to the guidewire 220 and/or the polymer sleeve 222 may be limited to less than ten percent of the length of the guidewire 220. In some embodiments, axial movement of the embolic protection device 230, the mounting sleeve 240, and/or the second mounting sleeve 250 along and/or relative to the guidewire 220 and/or the polymer sleeve 222 may be limited to less than five percent of the length of the guidewire 220. In some embodiments, axial movement of the embolic protection device 230, the mounting sleeve 240, and/or the second mounting sleeve 250 along and/or relative to the guidewire 220 and/or the polymer sleeve 222 may be limited to less than two percent of the length of the guidewire 220.

In some embodiments, the embolic protection system 200 may optionally include at least one stop 260 disposed on the guidewire 220 and/or the polymer sleeve 222, as seen in FIG. 6. In some embodiments, the at least one stop 260 may be fixedly attached to the guidewire 220 and/or the polymer sleeve 222. The at least one stop 260 may be configured to limit axial movement of the embolic protection device 230, the mounting sleeve 240, and/or the second mounting sleeve 250 along and/or relative to the guidewire 220 and/or the polymer sleeve 222 in a proximal direction, a distal direction, or both proximal and distal directions. In some embodiments, the at least one stop 260 may be positioned proximal of the mounting sleeve 240, distal of the mounting sleeve 240, proximal of the second mounting sleeve 250, distal of the second mounting sleeve 250, and/or between the mounting sleeve 240 and the second mounting sleeve 250.

In some embodiments, the at least one stop 260 may include a proximal stop 262 disposed on the guidewire 220 and/or the polymer sleeve 222 proximal of the mounting sleeve 240 and a distal stop 264 disposed distal of the mounting sleeve 240. In some embodiments, the at least one stop 260 may include a proximal stop 262 disposed on the guidewire 220 and/or the polymer sleeve 222 proximal of the mounting sleeve 240 and a distal stop 264 disposed distal of the second mounting sleeve 250. In some embodiments, the at least one stop 260 may further include an intermediate stop 266 disposed between the mounting sleeve 240 and the second mounting sleeve 250. Some suitable but non-limiting materials for the at least one stop 260, the proximal stop 262, the distal stop 264, and/or the intermediate stop 266, for example metallic and/or polymeric materials, are described below.

In some embodiments, the at least one stop 260 and/or the proximal stop 262 may be fixedly attached to the guidewire 220 and/or the polymer sleeve 222 proximate a proximal end of the polymer sleeve 222. In some embodiments, the at least one stop 260 and/or the distal stop 264 may be fixedly attached to the guidewire 220 and/or the polymer sleeve 222 proximate a distal end of the polymer sleeve 222. In some embodiments, the at least one stop 260 and/or the intermediate stop 266 may be fixedly attached to the guidewire 220 and/or the polymer sleeve 222 between the proximal end of the polymer sleeve 222 and the distal end of the polymer sleeve 222.

In some embodiments, the mounting sleeve 240 and/or a proximal end of the embolic protection device 230 may be axially constrained along and/or relative to the guidewire 220 and/or the polymer sleeve 222, and the second mounting sleeve 250 and/or a distal end of the embolic protection device 230 may be axially constrained along and/or relative to the guidewire 220 and/or the polymer sleeve 222. In some embodiments, the mounting sleeve 240 and/or a proximal end of the embolic protection device 230 may be axially constrained along and/or relative to the guidewire 220 and/or the polymer sleeve 222, and the second mounting sleeve 250 and/or a distal end of the embolic protection device 230 may be axially unconstrained along and/or relative to the guidewire 220 and/or the polymer sleeve 222. Leaving the second mounting sleeve 250 and/or the distal end of the embolic protection device 230 axially unconstrained may permit the second mounting sleeve 250 and/or the distal end of the embolic protection device 230 to stretch distally to provide a lower overall profile during sheathing, delivery, and/or retrieval.

The materials that can be used for the various components of the embolic protection system 100/200, the delivery catheter 110/210, the guidewire 120/220, the embolic protection device 130/230, the mounting sleeve 140/240, the second mounting sleeve 150/250, the at least one stop 160/260, etc. (and/or other systems disclosed herein) and the various elements thereof disclosed herein may include those commonly associated with medical devices. For simplicity purposes, the following discussion makes reference to the embolic protection system 100/200, the delivery catheter 110/210, the guidewire 120/220, the embolic protection device 130/230, the mounting sleeve 140/240, the second mounting sleeve 150/250, the at least one stop 160/260, etc. However, this is not intended to limit the devices and methods described herein, as the discussion may be applied to other elements, members, components, or devices disclosed herein, such as, but not limited to, the at least one support strut 132/232, the filter element 134/234, the proximal stop 162/262, the distal stop 164/264, the intermediate stop 166/266, the polymer sleeve 222, etc., and/or elements or components thereof.

In some embodiments, the embolic protection system 100/200, the delivery catheter 110/210, the guidewire 120/220, the embolic protection device 130/230, the mounting sleeve 140/240, the second mounting sleeve 150/250, the at least one stop 160/260, etc., and/or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. For example, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of the embolic protection system 100/200, the delivery catheter 110/210, the guidewire 120/220, the embolic protection device 130/230, the mounting sleeve 140/240, the second mounting sleeve 150/250, the at least one stop 160/260, etc., and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the embolic protection system 100/200, the delivery catheter 110/210, the guidewire 120/220, the embolic protection device 130/230, the mounting sleeve 140/240, the second mounting sleeve 150/250, the at least one stop 160/260, etc. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the embolic protection system 100/200, the delivery catheter 110/210, the guidewire 120/220, the embolic protection device 130/230, the mounting sleeve 140/240, the second mounting sleeve 150/250, the at least one stop 160/260, etc. to achieve the same result.

In some embodiments, a degree of Magnetic Resonance Imaging (MRI) compatibility is imparted into the embolic protection system 100/200, the delivery catheter 110/210, the guidewire 120/220, the embolic protection device 130/230, the mounting sleeve 140/240, the second mounting sleeve 150/250, the at least one stop 160/260, etc. For example, the embolic protection system 100/200, the delivery catheter 110/210, the guidewire 120/220, the embolic protection device 130/230, the mounting sleeve 140/240, the second mounting sleeve 150/250, the at least one stop 160/260, etc., and/or components or portions thereof, may be made of a material that does not substantially distort the image and create substantial artifacts (e.g., gaps in the image). Certain ferromagnetic materials, for example, may not be suitable because they may create artifacts in an MRI image. The embolic protection system 100/200, the delivery catheter 110/210, the guidewire 120/220, the embolic protection device 130/230, the mounting sleeve 140/240, the second mounting sleeve 150/250, the at least one stop 160/260, etc., or portions thereof, may also be made from a material that the MM machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-N® and the like), nitinol, and the like, and others.

In some embodiments, the embolic protection system 100/200, the delivery catheter 110/210, the guidewire 120/220, the polymer sleeve 222, the embolic protection device 130/230, the mounting sleeve 140/240, the second mounting sleeve 150/250, the at least one stop 160/260, etc., and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the embolic protection device 130/230, the filter element 134/234, etc. may include and/or be formed from a textile material. Some examples of suitable textile materials may include synthetic yarns that may be flat, shaped, twisted, textured, pre-shrunk or un-shrunk. Synthetic biocompatible yarns suitable for use in the present invention include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni-Co-Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun-types. The type and denier of the yarn chosen may be selected in a manner which forms a biocompatible and implantable prosthesis and, more particularly, a vascular structure having desirable properties.

In some embodiments, the embolic protection system 100/200, the delivery catheter 110/210, the guidewire 120/220, the embolic protection device 130/230, the mounting sleeve 140/240, the second mounting sleeve 150/250, the at least one stop 160/260, etc. may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vascoactive mechanisms.

It should be understood that this disclosure is, in many respects, only illustrative. Changes may be made in details, particularly in matters of shape, size, and arrangement of steps without exceeding the scope of the invention. This may include, to the extent that it appropriate, the use of any of the features of one example embodiment being used in other embodiments. The invention's scope is, of course, defined in the language in which the appended claims are expressed.

What is claimed is:

1. An embolic protection system, comprising:
a guidewire having a length, at least a portion of the guidewire having a non-circular cross-sectional shape; and
an embolic protection device including a mounting sleeve configured to attach the embolic protection device to the portion of the guidewire having the non-circular cross-sectional shape;
wherein the mounting sleeve includes a non-circular interior cross-sectional shape and a non-circular exterior cross-sectional shape.

2. The embolic protection system of claim 1, wherein the non-circular interior cross-sectional shape and the non-circular exterior cross-sectional shape are substantially identical.

3. The embolic protection system of claim 1, wherein the embolic protection device is slidable with respect to the guidewire.

4. The embolic protection system of claim 1, wherein the embolic protection device is non-rotatable with respect to the guidewire.

5. The embolic protection system of claim 1, further comprising a delivery catheter having a lumen extending therethrough.

6. The embolic protection system of claim 5, wherein the embolic protection device is disposed within a distal portion of the lumen in a delivery configuration.

7. The embolic protection system of claim 6, wherein the embolic protection device is expandable to a deployed configuration when the embolic protection device is disposed outside of the lumen.

8. The embolic protection system of claim 1, wherein the embolic protection device includes a second mounting sleeve configured to attach to the portion of the guidewire having the non-circular cross-sectional shape.

9. The embolic protection system of claim 8, wherein the second mounting sleeve includes the non-circular interior cross-sectional shape of the mounting sleeve and the non-circular exterior cross-sectional shape of the mounting sleeve.

10. The embolic protection system of claim 9, wherein the mounting sleeve is fixed to the embolic protection device proximate a proximal mouth of the embolic protection device and the second mounting sleeve is fixed to the embolic protection device proximate a distal end of the embolic protection device.

11. The embolic protection system of claim 10, wherein the mounting sleeve and the second mounting sleeve define a distance from a proximal end of the mounting sleeve to a distal end of the second mounting sleeve;
wherein the portion of the guidewire having the non-circular cross-sectional shape is longer than the distance from the proximal end of the mounting sleeve to the distal end of the second mounting sleeve and less than the length of the guidewire.

12. The embolic protection system of claim 1, further comprising a proximal stop disposed on the guidewire proximal of the mounting sleeve and a distal stop disposed on the guidewire distal of the mounting sleeve.

13. The embolic protection system of claim 12, wherein the proximal stop is fixedly attached to the guidewire proximate a proximal end of the portion of the guidewire having the non-circular cross-sectional shape.

14. The embolic protection system of claim 12, wherein the distal stop is fixedly attached to the guidewire proximate a distal end of the portion of the guidewire having the non-circular cross-sectional shape.

15. The embolic protection system of claim 1, wherein the embolic protection device includes a filter element and at least one support strut.

16. An embolic protection system, comprising:
a guidewire having a length, at least a portion of the guidewire having a polygonal cross-sectional shape; and
an embolic protection device including a mounting sleeve configured to attach the embolic protection device to the portion of the guidewire having the polygonal cross-sectional shape;
wherein the mounting sleeve includes a polygonal interior cross-sectional shape and a polygonal exterior cross-sectional shape.

17. The embolic protection system of claim 16, wherein the polygonal interior cross-sectional shape and the polygonal exterior cross-sectional shape are substantially identical.

18. The embolic protection system of claim 16, wherein the embolic protection device includes a second mounting sleeve configured to attach to the portion of the guidewire having the polygonal cross-sectional shape.

19. The embolic protection system of claim 18, wherein the second mounting sleeve includes the polygonal interior cross-sectional shape of the mounting sleeve and the polygonal exterior cross-sectional shape of the mounting sleeve.

20. An embolic protection system, comprising:
a guidewire having a length, at least a portion of the guidewire having a non-circular cross-sectional shape; and
an embolic protection device including a filter element, a mounting sleeve, and a second mounting sleeve;
wherein the mounting sleeve and the second mounting sleeve are configured to slidably attach the embolic protection device to the portion of the guidewire having the non-circular cross-sectional shape;
wherein the mounting sleeve and the second mounting sleeve each include a non-circular interior cross-sectional shape and a non-circular exterior cross-sectional shape;
wherein axial movement of the embolic protection device relative to the guidewire is limited to less than ten percent of the length of the guidewire.
